(19) United States
(12) Patent Application Publication
(10) Pub. No.: US 2006/0278235 A1
(43) Pub. Date: Dec. 14, 2006

(54) Tracheal tube with above the cuff drainage

(76) Inventors: Steven C. White, Orono, MN (US); Roger Johnson, Mercer Island, WA (US); Paul Harmon, Edina, MN (US); Bruce Wigness, Minneapolis, MN (US)

Correspondence Address: POPOVICH, WILES & O'CONNELL, PA;650 THIRD AVENUE SOUTH, SUITE 600, MINNEAPOLIS, MN, 55402, US

(21) Appl. No.: 11/152,310

(22) Filed: Jun. 14, 2005

(51) Int. Cl. A62B 9/06; A61M 16/00

(52) U.S. Cl. 128/207.14; 128/200.26; 128/207.15; 128/207.16

(57) ABSTRACT

A system comprising an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated.

### FIELD OF THE INVENTION

The present invention relates to tracheal tubes used during ventilation of a patient.

### BACKGROUND OF THE INVENTION

When a patient is unable to adequately breathe independently, an external mechanical ventilator may be used to provide temporary or permanent breathing support. The ventilator pumps air into and out of the subject's lungs such as, for example, through an endotracheal (ET) tube or other tracheal tube. In one example, a distal portion of the tracheal tube is introduced via the subject's mouth. A proximal portion of the endotracheal tube is connected to the ventilator. An inflatable cuff near the distal end of the endotracheal tube is inflated to completely occupy the intratracheal region surrounding the endotracheal tube. This creates a seal that prevents airflow through the trachea other than through the endotracheal tube that the ventilator can provide the subject with breathing support through the endotracheal tube.

Fluid accumulates below the cuff of the endotracheal tube. One technique for removing accumulated fluid from the lungs (below the cuff) includes interrupting the patient's ventilation by disconnecting the proximal end of the endotracheal tube from the ventilator. A suction tube is then inserted through the endotracheal tube beyond the cuff at its distal end. By applying an airflow-creating vacuum to the proximal end of the suction tube, fluid is removed from the lungs.

Fluid also accumulates above the cuff of the endotracheal tube. In a long term intubation subject, pathogenic bacteria multiply in the pool of secretions that accumulate above the inflated endotracheal tube cuff and can cause pneumonia. For this reason, it is desirable to construct endotracheal tubes that incorporate a device to draw fluids from above the cuff.

Tracheal tubes are described in U.S. Patent Publication No. US 2003/0145860 A1, published Aug. 7, 2003, entitled “Surface Energy Assisted Fluid Transport System”, and the contents of this application are hereby incorporated by reference herein.

The invention provides an improved tracheal tube having above the cuff drainage.

### SUMMARY OF THE INVENTION

The invention provides a system comprising: an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated.

The invention provides a system comprising: an elongated member, sized and shaped to be inserted within a subject's trachea, the elongated member comprising proximal and distal ends; a seal, extending around the elongated member at or near the distal end of the elongated member; at least one fluid pickup port, positioned near the distal end of the elongated member, the at least one fluid pickup port located more proximal than at least a portion of the seal; and at least one lumen, coupled in fluid communication with the at least one fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated.

The invention provides a system comprising: an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, the inflatable cuff being cup-shaped and oriented so that the proximal surface directs fluid toward the at least one wicking fluid pickup port.

The invention provides a method comprising: inserting into a subject's trachea an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated, and wherein the elongated member comprises an airflow lumen extending longitudinally from at or near the distal end of the elongated member to or near the proximal end of the elongated member;

inflating the inflatable cuff to obstruct airflow at a first location outside of the elongated member and inside the trachea;

ventilating at least one of the subject's lungs through the elongated member;

wicking fluid, at a location that is more proximal than the first location; and

drawing the wicked fluid out of the subject.

Additional features and advantages of the invention are set forth in the description which follows and in part will be apparent from the description. The objectives and other advantages of the invention will be realized and attained by the tracheal tube having above the cuff drainage as particularly pointed out in the written description and claims.

It is to be understood that both the foregoing general description and the following detailed description are exemplary and explanatory and are intended to provide further explanation of the invention as claimed.

### BRIEF DESCRIPTION OF THE DRAWINGS

FIG. 1A is a perspective view of one example of a system including an endotracheal tube assembly and a pump assembly.

FIG. 1B is a cross-sectional view of the system of FIG. 1A showing a distal portion of the endotracheal tube assembly inserted within a portion of a patient's trachea.

FIG. 1C is a perspective view of the system of FIG. 1A.

FIG. 2 is a flow chart of one example of operation of portions of the system for removing mucus during mechanical ventilation of a patient using the endotracheal tube assembly.

FIG. 3 is a cross-sectional view of one example of a distal portion of the endotracheal tube assembly inserted within a portion of a patient's trachea.

FIG. 4A is a cross-sectional view of one example of a distal portion of the endotracheal tube assembly inserted within a portion of a patient's trachea.

FIG. 4B is a cross-sectional view of FIG. 4A.

FIG. 4C is a cross-sectional view of FIG. 4A.

FIG. 4D is a top perspective view of FIG. 4A.

FIG. 5A is a cross-sectional view of one example of a distal portion of the endotracheal tube assembly inserted within a portion of a patient's trachea.

FIG. 5B is a cross-sectional view of FIG. 5A.

FIG. 5C is a cross-sectional view of FIG. 5A.

FIG. 6 is a cross-sectional view of one example of a distal portion of the endotracheal tube assembly inserted within a portion of a patient's trachea.

FIG. 7A is a cross-sectional view of one example of a distal portion of the endotracheal tube assembly inserted within a portion of a patient's trachea.

FIG. 7B is a cross-sectional view of FIG. 7A.

FIG. 8 is a perspective view of one embodiment of a fluid pickup port.

FIG. 9A is a perspective view of one example of a distal portion of the endotracheal tube assembly inserted within a portion of a patient's trachea.

FIG. 9B is a cut-away perspective view of FIG. 9A.

FIG. 9C is a top view of FIG. 9A.

FIG. 10 is a cross-sectional view of one example of a distal portion of an endotracheal tube assembly.

### DETAILED DESCRIPTION OF THE INVENTION

The invention provides a system comprising: an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated. In one embodiment, the at least one wicking fluid pickup port is disposed from 0.2 to 2 mm from the inner tracheal wall when the distal end of the elongated member is positioned within a trachea of a person and the inflatable cuff is inflated. In another embodiment, the at least one wicking fluid pickup port is disposed immediately adjacent to the inner tracheal wall when the distal end of the elongated member is positioned within a trachea of a person and the inflatable cuff is inflated.

In one embodiment, the at least one wicking fluid pickup port is disposed immediately above the proximal surface of the cuff. In another embodiment, the at least one wicking fluid pickup port is disposed on the proximal surface of the cuff. In one embodiment, at least a portion of the proximal surface is slanted relative to a longitudinal axis of the elongated member to direct fluid toward the at least one wicking fluid pickup port. In another embodiment, the entire proximal surface of the inflatable cuff is slanted relative to a longitudinal axis of the elongated member to direct fluid toward the at least one wicking fluid pickup port. In one embodiment, the entire proximal surface of the cuff and the longitudinal axis of the elongated member meet at an angle of less than 90 degrees. In another embodiment, the entire proximal surface of the cuff and the longitudinal axis of the elongated member meet at an angle of less than 80 degrees.

In one embodiment, the at least a portion of the proximal surface slanted relative to a longitudinal axis of the elongated member forms a V-shaped depression. In one embodiment, the entire proximal surface of the inflatable cuff is slanted relative to a longitudinal axis of the elongated member to direct fluid toward the at least one wicking fluid pickup port and the proximal surface comprises a V-shaped depression to direct fluid toward the at least one wicking fluid pickup port.

In one embodiment, the cuff comprises a reinforcing element. In another embodiment, the cuff comprises a semi-rigid polymer. In another embodiment, the cuff is made of a semi-rigid polymer.

In one embodiment, a portion of the inflatable cuff is made of a semi-elastic material that expands only a predetermined amount. In another embodiment, the predetermined amount of expansion places the wicking fluid pickup port a predetermined distance from the tracheal wall when the cuff is inflated. In one embodiment, the semi-elastic material is a woven or non-woven fabric.

In one embodiment, the elongated member comprises an airflow lumen extending longitudinally from at or near the distal end of the elongated member to or near the proximal end of the elongated member. In another embodiment, the airflow lumen is sized to provide adequate ventilation to at least one lung of a person when the airflow lumen is coupled, at or near the proximal end of the elongated member, to a mechanical ventilator.

In one embodiment, the system further comprises a cuff lumen, coupled in fluid communication with the inflatable bladder, the cuff lumen extending longitudinally to or near the proximal end of the elongated member. In another embodiment, the elongated member comprises a hollow tube including a ventilation airflow lumen extending between the distal end of the elongated member and the proximal end of the elongated member, and in which at least a portion of the cuff lumen extends longitudinally through a sidewall portion of the hollow tube. In one embodiment, the elongated member comprises a hollow tube including a ventilation airflow lumen extending between the distal end of the elongated member and the proximal end of the elongated member, and in which at least a portion of the cuff lumen extends longitudinally within the ventilation airflow center lumen. In another embodiment, the elongated member comprises a hollow tube including a ventilation airflow lumen extending between the distal end of the elongated member and the proximal end of the elongated member, and in which at least a portion of the cuff lumen extends longitudinally outside the hollow tube.

In one embodiment, the inflatable cuff comprises a single inflatable bladder. In another embodiment, the inflatable cuff comprises two inflatable bladders. In one embodiment, the two inflatable bladders are coupled in fluid communication to first and second cuff lumens, the first and second cuff lumens extending longitudinally to or near the proximal end of the elongated member. In another embodiment, a first inflatable bladder is coupled in fluid communication to a cuff lumens, the lumen extending longitudinally to or near the proximal end of the elongated member, and the second inflatable bladder is coupled in fluid communication with the first inflatable bladder by one or more pressure relief valves that open when the pressure in the first inflatable bladder exceeds a certain pressure.

In one embodiment, the at least one wicking fluid pickup port includes at least one of a size, shape, and material characteristic that obtains a surface energy capable of assisting in introducing mucus into the at least one wicking fluid pickup port. In another embodiment, the system further includes a pump coupled in fluid communication with the at least one lumen that is in fluid communication with the at least one wicking fluid pickup port. In another embodiment, at least one lumen that is in fluid communication with the at least one wicking fluid pickup port comprises a portion that extends within an interior portion of the inflatable cuff. In one embodiment, a portion of the at least one lumen that is in fluid communication with the at least one wicking fluid pickup extends distally past the at least one wicking fluid pickup port. In another embodiment, the pump comprises a peristalsis pump.

In one embodiment, the system further includes a holding receptacle coupled in fluid communication with the at least one lumen that is in fluid communication with the at least one wicking fluid port. In another embodiment, the elongated member is sized and shaped to be inserted through an airflow passage of a tracheal tube assembly to a desired bronchial tube of the subject. In one embodiment, the wicking fluid pickup port has a rounded tip. In another embodiment, the elongated member comprises a reference mark to assist in proper placement of the elongated member.

In one embodiment, the system comprises two or more wicking fluid pickup ports. In another embodiment, the lumen coupled to the at least one wicking fluid pickup port exits the inflatable cuff distal of the proximal surface of the inflatable cuff. In one embodiment, the at least one wicking fluid pickup port is disposed immediately above or on the proximal surface of the cuff and an area of the cuff surrounding the at least one wicking fluid pickup port is hydrophilic. In another embodiment, an area of the cuff distant from the at least one wicking fluid pickup port is hydrophobic.

The invention provides a system comprising: an elongated member, sized and shaped to be inserted within a subject's trachea, the elongated member comprising proximal and distal ends; a seal, extending around the elongated member at or near the distal end of the elongated member; at least one fluid pickup port, positioned near the distal end of the elongated member, the at least one fluid pickup port located more proximal than at least a portion of the seal; and at least one lumen, coupled in fluid communication with the at least one fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated. In one embodiment, the system further comprises a vacuum pump coupled in fluid communication with the at least one lumen that is in fluid communication with the at least one wicking fluid pickup port.

The invention provides a system comprising: an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, the inflatable cuff being cup-shaped and oriented so that the proximal surface directs fluid toward the at least one wicking fluid pickup port.

The invention provides a method comprising:

inserting into a subject's trachea an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated, and wherein the elongated member comprises an airflow lumen extending longitudinally from at or near the distal end of the elongated member to or near the proximal end of the elongated member;

inflating the inflatable cuff to obstruct airflow at a first location outside of the elongated member and inside the trachea;

ventilating at least one of the subjects lungs through the elongated member;

wicking fluid, at a location that is more proximal than the first location; and

drawing the wicked fluid out of the subject. In one embodiment, the drawing the wicked fluid out of the subject includes using a peristalsis pump to provide a pressure for drawing the wicked fluid out of the subject. In one embodiment, the drawing the wicked fluid out of the subject includes matching a flow rate at which the wicked fluid is drawn out of the subject to a mucus generation rate of the subject.

In one embodiment of the invention, the area immediately surrounding the fluid pickup port is made of a material that attracts fluids or is treated to attract fluids (i.e., the area is hydrophilic). Polymers that attract tracheal secretions include polyphenylene oxide, polyethylene terephthalate, polyamide, polyimide, and polyether block amide. In another embodiment of the invention, the area immediately surrounding the fluid pickup port is made of a material that attracts fluids or is treated to attract fluids (i.e., the area is hydrophilic) and the rest of the cuff is treated to repel fluids (i.e., the rest of the cuff is hydrophobic).

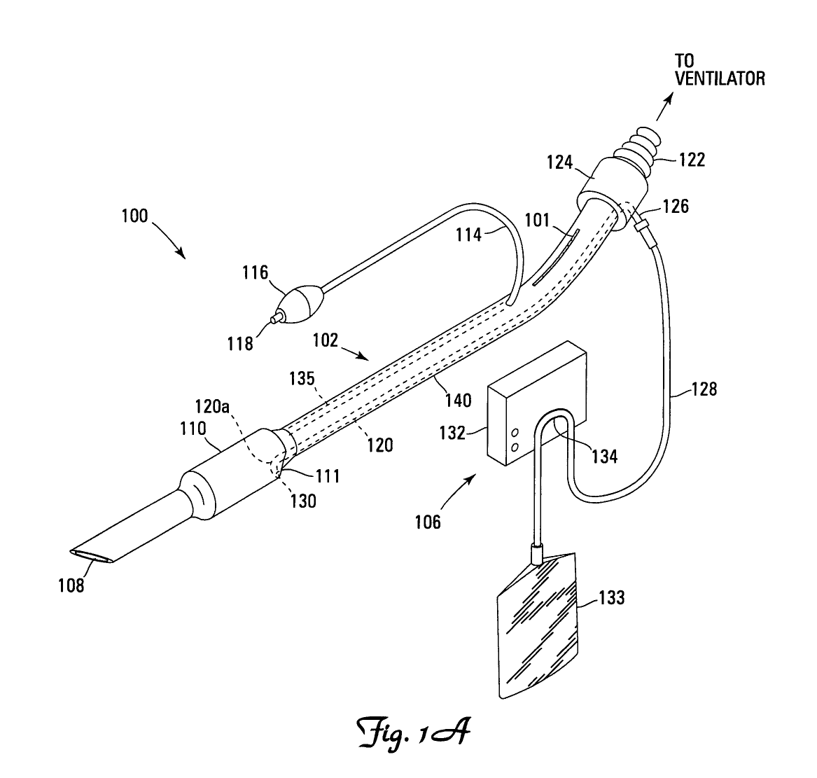

FIG. 1A is a perspective view illustrating generally, by way of example, but not by way of limitation, one embodiment of a system 100 that includes one example of an endotracheal tube assembly 102, and a pump assembly 106 coupled to a location that is at or near a proximal end of endotracheal tube assembly 102. In the example of FIG. 1A, endotracheal tube assembly 102 includes an endotracheal tube 140 and an air passage 108, extending longitudinally between the distal and proximal ends of endotracheal tube assembly 102. A bladder-like inflatable cuff 110 (or other seal) is located about the outer circumference of endotracheal tube assembly 102 near its distal end. The fluid pickup port 130 is disposed on the inflatable cuff 110 and transports fluid through fluid removal lumen 120. The distal portion of fluid removal lumen 120 extends into the inflatable cuff 110 (the distal portion of the fluid removal lumen that extends into the inflatable cuff is designated 120a) and terminates in fluid pickup port 130, which is located on the proximal surface 112 in the proximal V-shaped depression 111 of the inflatable cuff 110. In use, the proximal V-shaped depression 111 is located on the lower side of the supine patient. To help in the placement of the proximal V-shaped depression 111, a reference mark 101 is provided on endotracheal tube assembly 102. The reference marking can appear anywhere on the endotracheal tube assembly. The proximal V-shaped depression 111 directs fluid to the fluid pickup port 130. The fluid pickup port 130 is adjacent to the wall of the trachea 150, when the inflatable cuff 110 is inflated. See FIG. 1B. A cuff lumen 135 extends through endotracheal tube assembly 102 from cuff 110 to the proximal portion of endotracheal tube assembly 102. For example, cuff lumen 135 may run integrally within a wall of endotracheal tube assembly 102, as a separate tube extending through air passage 108, or outside the wall of the endotracheal tube assembly 102.

In this example, at the proximal end portion of endotracheal tube assembly 102, cuff lumen 135 is coupled in fluid communication with external cuff tube 114, which extends outwardly therefrom toward cuff pressure bladder 116 and inflation port 118, or similar pump device for inflating cuff 110. Cuff 110 is capable of being inflated when endotracheal tube assembly 102 is disposed within a lumen (e.g., within a patient's trachea). Inflating cuff 110 provides a seal that ensures that airflow occurs within air passage 108, rather than through the trachea outside endotracheal tube assembly 102. In one example, cuff 110 is inflated by introducing air into inflation port 118 by using a syringe, and by then compressing cuff pressure bladder 116 to force the air through external cuff tube 114 and cuff lumen 135 into cuff 110.

The proximal end of endotracheal tube assembly 102 terminates at an end connector 124. In this example, end connector 124 is sized and shaped to allow coupling to a ventilator tube 122, which, in turn, is coupled to a mechanical lung ventilator. End connector 124 provides fluid communication between ventilator tube 122 and air passage 108 of endotracheal tube assembly 102.

In the example of FIG. 1A, fluid pickup port 130 is located near the distal end of endotracheal tube assembly 102 and at the V-shaped depression 111 of the inflatable cuff 110. See FIGS. 1B and 1C. Therefore, in this example, when the distal end of endotracheal tube assembly 102 is introduced into a patient's trachea, fluid pickup port 130 is located within the patient at the V-shaped depression 111 of the inflatable cuff 110. A fluid removal lumen 120 extends and provides fluid communication between fluid pickup port 130 and a coupling stem 126 (located at or near the proximal end of endotracheal tube assembly 102) or a like coupling device. A fluid removal tube 128 is coupled in fluid communication with coupling stem 126, for further carrying the fluid being removed to a bag or other holding receptacle 133. In this example, pump assembly 106 includes a constant volume (CV) or other low volume pump 132, having a pump head 134 coupled to a portion of fluid removal tube 128 for providing a negative pressure within fluid removal tube 128. This assists in drawing a liquid column or mixture of liquid and air through fluid removal tube 128 to holding receptacle 133.

In this example, the fluid pickup port 130 wicks fluid into the port 130. That is, the fluid pickup port 130 is sized, shaped, made of a particularly selected material, and/or otherwise configured to use interfacial surface energy (also referred to as surface tension) to introduce a bodily or other fluid (such as mucus or the like) into the wicking fluid pickup port 130. Interfacial surface energies cause a resulting “skin” to form (or, conversely, a repulsion to occur) at an air/liquid interface boundary. Similarly, an attraction or repulsion between a liquid fluid and its interface boundary with a solid may result because of its interfacial surface energy. This interfacial edge effect can provide a capillary action whereby a liquid is pulled into a small pipe, i.e., a capillary. The relative value of the surface energy of the solid wall and that of the liquid determines whether the liquid is more attracted to the wall (in which case a “wicking” occurs which pulls the fluid to the wall) or to itself (in which case it avoids “wetting” the wall). In the present case, the relative value of the surface energy will be affected by, among other things, the size of the fluid pickup port 130, the shape of the fluid pickup port 130, and the material characteristics of the endotracheal tube assembly 102 in which the fluid pickup port 130 is formed, and the characteristics of the air/fluid interface.

In the example of FIGS. 1A and 1B, endotracheal tube assembly 102 is designed to use the interfacial surface energy to draw the mucus or the like into the fluid pickup port 130. In the example of FIGS. 1A and 1B, once the mucus, secretions, or the like pulls itself into the fluid pickup port 130 using the surface energy effect, it is then subjected to a negative pressure, such as that generated by remote external constant volume pump 132, to draw such fluid toward holding receptacle 133.

In this example, fluid removal lumen 120, coupling 126, and fluid removal tube 128 are each sized, shaped, made of a particularly selected material, or otherwise configured such that the surface energy of the mucus (or similar bodily fluid) causes a “skin” to bridge the entire interior cross section of the conduit formed by these components. As a result, mucus, secretions, like fluid, and/or air bubbles are pulled by pump 132 through the conduit provided by these components. By contrast, conventional airflow-based vacuum devices generally pull liquid fluid by using a large ratio of entrapping air (or other gaseous substance) to the liquid fluid being entrapped by the air. This is because such airflow-based vacuum devices typically depend on the air movement at the intake port to draw the fluid into the port, rather than using surface energy to draw fluid (i.e., “wick” the fluid) into the intake port.

Although not required, in one example, the pressure provided by pump 132 is adjusted to remove fluid at a desired steady-state rate that is selected such that the extracted material passing through the conduit provided by fluid removal lumen 120, coupling 126, and fluid removal tube 128 is almost all liquid (including, among other things, viscous liquids and liquid suspensions bearing suspended solids and/or entrapped gas bubbles), rather than a liquid in combination with a more than insubstantial amount of air or other gaseous substance. This results from the wicking of the mucus or like fluid into the fluid pickup port 130 using surface energy. Similarly, the degree of wicking provided by the fluid pickup port 130 can be adjusted to match or approximate the subject's mucus generation rate. The present systems and methods of mucus removal may (but need not) be provided concurrent to the ventilation of the patient, such as continuously.

Thus, in this example, pump 132 provides a negative pressure such that entrapment of fluid by airflow is not required to transport the fluid toward holding receptacle 133. A peristalsis pump is only one example of a constant volume (CV) pump capable of supplying a negative pressure against the fluid. Alternative embodiments may use one or more other types of low volume pumps, which need not be CV pumps, and which may be operated intermittently. Some other pump examples include, among other things, an accordion-style cavity with one-way valves for intake and discharge, such that repeated compressing of the cavity transports the fluid.

In one example, at least a portion of the conduit provided by fluid removal lumen 120, coupling 126, and fluid removal tube 128 (at least up to pump head 134) is designed in material and size such that liquid fluid being transported can span the inside diameter of said conduit. The design is such that any air bubbles introduced at the fluid pickup port 130 preserve an intact air/liquid “skin” or “bridge” that spans the inside diameter of said conduit. As a result, such air bubbles can be conceptualized as being carried along by the liquid column being transported as if they were a part of that liquid column. Therefore, entrapment by high airflow is not required or used to obtain the desired mucus removal. The components forming the conduit are sufficiently rigid to prevent their collapse under the pressures used to move the fluid up against gravity and to overcome the viscosity and holding power of any fluid bridging the fluid pickup port 130.

In one embodiment, the inner diameter of at least a portion fluid removal lumen 120 is sized so as to be small enough to permit it to be bridged by the fluid/air “skin” as a result of the interfacial surface tension. The corresponding size of the inner diameter of fluid removal lumen 120 can be conceptually approximated using the following Equation 1: h=(2·y·cos Θ_{c})÷(r·Pₑ·g). Equation 1 illustrates that, to obtain the desired bridging, the inner diameter of fluid removal lumen 120 must be small enough such that a column of the liquid of interest (e.g., mucus) can be lifted by surface energy to a height just greater than the height, h, of the meniscus. In Equation 1, y is the surface tension value of the fluid, Θ_{c} is the angle at which the fluid contacts the inner circumference of the fluid removal lumen 120, r is the inner radius of the fluid removal lumen 120, Pₑ is the fluid density in air, and g is the acceleration due to gravity. Thus, in one example, the size of the inner diameter of fluid removal lumen 120 is increased until h equals the height of the meniscus, as illustrated in Equation 1. Similarly, the size of the inner diameter of the wicking fluid pickup port 130 is determined as described with respect to Equation 1.

In another embodiment of the invention, the fluid pickup port 130 does not exhibit significant wicking action and the fluid is removed by a vacuum pump. In this embodiment, low volume pump 132 is replaced with a vacuum pump. In another embodiment, the fluid removal lumen 120 can be a sleeve lumen in the form of an annulus.

FIG. 1B is a cross-sectional view illustrating generally, by way of example, but not by way of limitation, one embodiment of a distal portion of endotracheal tube assembly 102 inserted within a portion of a patient's trachea 150. The inner wall 152 of the patient's trachea 150 is coated with mucus 154. Cuff 110 is illustrated, in this example, as having been inflated to seal trachea 150. In this example, the fluid pickup port 130 is connected to fluid removal lumen 120, which, in this example, extends longitudinally within the wall 208 of endotracheal tube 140 toward its proximal end. However, in another example, one or more fluid removal lumens 120 extend as a tube running longitudinally through air passage 108. In yet another example, one or more fluid removal lumens 120 extend as a tube running along an exterior portion of endotracheal tube assembly 102.

A different number of fluid removal lumens 120 may be provided, for example, corresponding to a different number of fluid pickup ports 130. This increases the number of surface energy assisted mucus collection sites. Such fluid pickup ports may be located in many different possible configurations. In one such example, system 100 includes a single fluid pickup port 130 and a corresponding single fluid removal lumen 120.

In one example, one or more of fluid pickup ports 130 is designed to allow it to act as a safety vent for another of fluid pickup ports 130. In an alternative example, a separate safety vent port is provided, rather than using one of the fluid pickup ports 130 as a safety vent port. This may be advantageous in tailoring the safety pressure value of the safety vent port.

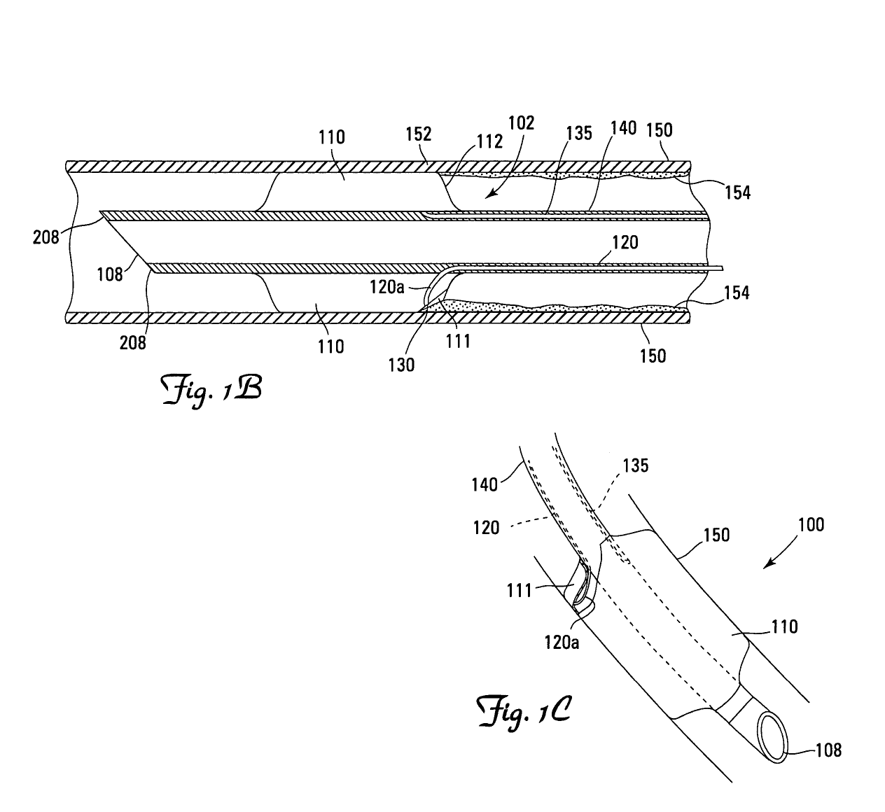

FIG. 2 is a flow chart illustrating generally, by way of example, but not by way of limitation, one embodiment of operating portions of system 100 for removing mucus during mechanical ventilation of a patient using an endotracheal tube assembly 102. At 1202, endotracheal tube assembly 102 is then inserted into trachea 200. At 1204, end connector 124 of endotracheal tube assembly 102 is coupled to the mechanical ventilator. At 1206, pump assembly 106 and holding receptacle 133 are connected to endotracheal tube assembly 102, such as by connecting at least one fluid removal tube 128 to coupling 126. In one example, holding receptacle 133 includes a waste bag. The waste bag is initially collapsed. The waste bag will expand with the collected mucus and any accumulated air bubbles that are discharged by pump 132. At 1208, once the endotracheal tube assembly 102 is in place for a short period of time, the mucus 154 on the inner wall 152 of trachea 150 will wick onto and then into the fluid pickup port 130. At 1210, pump 132 is turned on. This creates a negative pressure in the conduit. As a result, the wicked-in mucus and other secretions are transported through the conduit toward holding receptacle 133. In one example, at 1212, the flow rate of the mucus and other secretions is selected such that it approximately matches the mucus and other secretion generation rate. This avoids mucus and other secretions accumulating above cuff 110 by using too low of a flow rate. This also avoids filling holding receptacle 133 with air by using too high of a flow rate. This also preserves the bridging skin of the liquid mucus and other secretions across the fluid pickup port 130, or across a safety vent or the like, such as discussed elsewhere in this document.

In one operational variation, the direction of fluid transport through the conduit is reversed, such as for introducing medicine and/or irrigation fluid or the like through the conduit and out of the fluid pickup port 130. For example, delivery of irrigation fluid to the pickup area within trachea 150 may aid in softening hardened mucus, or even in dissolving mucus castings. Therefore, system 100 is adapted to accommodate mucus of different consistencies.

In one example, the medicine, irrigation fluid, or the like is introduced by swapping in a different holding receptacle 133 (carrying the drug, irrigation fluid, or the like) and reversing the direction of pump 132. In another example, a different holding receptacle and/or pump is used for fluid delivery to the patient.

In one example, the medicine and/or irrigation fluid or the like has a different surface energy characteristic from the mucus for which the fluid transport conduit and pickup port 130 were designed. Under certain such circumstances, therefore, the medicine and/or irrigation fluid or the like is not retained within the conduit by the wicking. Therefore, such medicine and/or irrigation fluid may be delivered out of the same pickup port 130 that wicks-in mucus.

In another variation, in which the patient's lungs are irrigated by a medicinal or other irrigation fluid (either using system 100, or otherwise), system 100 is used to remove excess irrigation fluid using one or more fluid pickup ports 130 that are particularly designed to wick in the irrigation fluid. In one such example, the irrigation fluid is introduced and removed through different ports, which are tailored to provide these different functions.

In another example, the surface energy characteristics of the at least one pickup port 130 and/or the conduit are changed during the introduction of the medicine and/or irrigation fluid or the like. In one example, a temporary modulation of the surface energy at a particular location (e.g., within at least one pickup port 130 or within one or more portions of the fluid transport conduit) may be obtained by introducing a surfactant. In another example, at least one electrode (e.g., at or near the at least one pickup port 130) modulates a local surface energy characteristic and/or provides an electric field that assists in expelling a drug or other fluid out of the at least one pickup port 130. In a further example, an electric field is applied to the electrode to adjust the rate at which the drug is introduced into the patient. In one example, the electrode is located at or near the at least one pickup port 130, and is connected to a wire that extends longitudinally through endotracheal tube assembly 102, from at or near its distal end to at or near its proximal end, for coupling the electrode to an external electrical energy source.

Modifying the surface energy characteristic at the at least one pickup port 130 and/or within the fluid transport conduit is not restricted to the above example of introducing a drug, fluid, or the like into a patient. In one example, the surface energy characteristics varies at one or more different locations of the at least one pickup port 130 and along the fluid transport conduit. Such variations are obtained, in one example, by varying the size, shape, and/or material characteristics at these one or more different locations. Moreover, a needed change in lumen size at a particular location in the at least one pickup port 130 or the fluid transport conduit may be offset, if needed, by a corresponding change in another surface tension affecting characteristic (e.g., material property, embedded electrode, etc.) at that location to preserve the bridging or sealing action, at that location, of the fluid being transported. In another example, a change in a surface tension affecting characteristic is used to preserve a spanning fluid/air interface bridge or to otherwise accommodate a branching or other junction of fluid transportation lumens, such as wherein an increased diameter is desired.

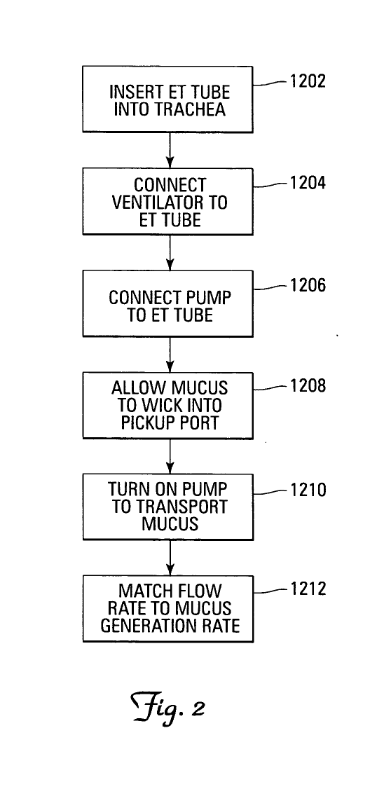

FIG. 3 shows an endotracheal tube 140 similar to that shown in FIG. 1B, except that the distal portion of the fluid removal lumen that extends into the inflatable cuff that is designated 120a extends further distally inside the inflatable cuff 110.

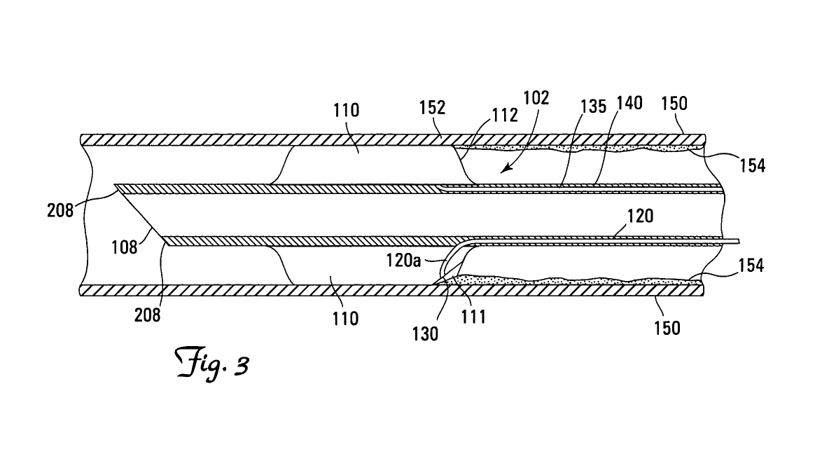

FIG. 4A shows an endotracheal tube 140 that is similar to that shown in FIG. 1B, except that the fluid removal lumen 120 does not extend into the inflatable cuff 110a, 110b, and the inflatable cuff comprises two inflatable portions 110a and 110b. The reason for providing two inflatable portions is to insure that the cuff inflates without wrinkles or creases near the inflation port. Wrinkles or creases could allow fluid to pass by the cuff and not be removed through the pickup port 130. Because inflatable portion 110a inflates before inflatable portion 110b, any wrinkles or creases will be displaced to inflatable portion 110b. Cuff portion 110a preferably is inflated first and then cuff portion 110b is inflated. This method of inflation insures that fluid pickup port 130 is properly positioned relative to the inner wall 152 of the patient's trachea.

The distal portion of fluid removal lumen 120 exits the wall 208 of the endotracheal tube 140 above the inflatable cuff 110a (the distal portion of the fluid removal lumen that extends from the wall 208 of the trachea tube is designated 120a) and terminates in fluid pickup port 130, which is located immediately above the proximal portion of the inflatable cuff 110a. The fluid pickup port 130 is adjacent to the wall of the trachea 150 (when the inflatable cuff 110 is inflated as shown). The inner wall 152 of the patient's trachea 150 is coated with mucus 154.

A cuff lumen 135a extends through endotracheal tube assembly 102 from cuff 110a to the proximal portion of endotracheal tube assembly 102. A cuff lumen 135b extends through endotracheal tube assembly 102 from cuff 110b to the proximal portion of endotracheal tube assembly 102.

In this example, the fluid port 130 is connected to fluid removal lumen 120, which, in this example, extends longitudinally within the wall 208 of endotracheal tube 140 toward its proximal end.

A different number of fluid removal lumens 120 may be provided, for example, corresponding to a different number of fluid pickup ports 130. This increases the number of surface energy assisted mucus collection sites. Such fluid pickup ports may be located in many different possible configurations.

In the example of FIG. 4A, fluid pickup port 130 is located near the distal end of endotracheal tube assembly 102 and above the V-shaped depression 111 of the inflatable cuff 110a. Therefore, in this example, when the distal end of endotracheal tube assembly 102 is introduced into a patient's trachea, fluid pickup port 130 is located within the patient at the V-shaped depression 111 of the inflatable cuff 110a. In this example, the fluid pickup port 130 wicks fluid into the port 130. In another embodiment of the invention, the fluid pickup port 130 does not exhibit significant wicking action and the fluid is removed by a vacuum pump. In this embodiment, low volume pump 132 is replaced with a vacuum pump.

FIG. 4B is a cross section of FIG. 4A. FIG. 4C is a cross section of FIG. 4A and shows the positions of inflatable cuff portions 110a and 110b. FIG. 4D is a top perspective view showing the V-shaped depression 111 that directs fluid to the pickup port 130. The pickup port 130 can have a beveled or non-beveled end. A beveled end could provide a smoother surface in some embodiments. Distal portion 120a can be attached to the cuff 110 by an adhesive. Using both an adhesive and a beveled end can provide a smoother surface in some embodiments.

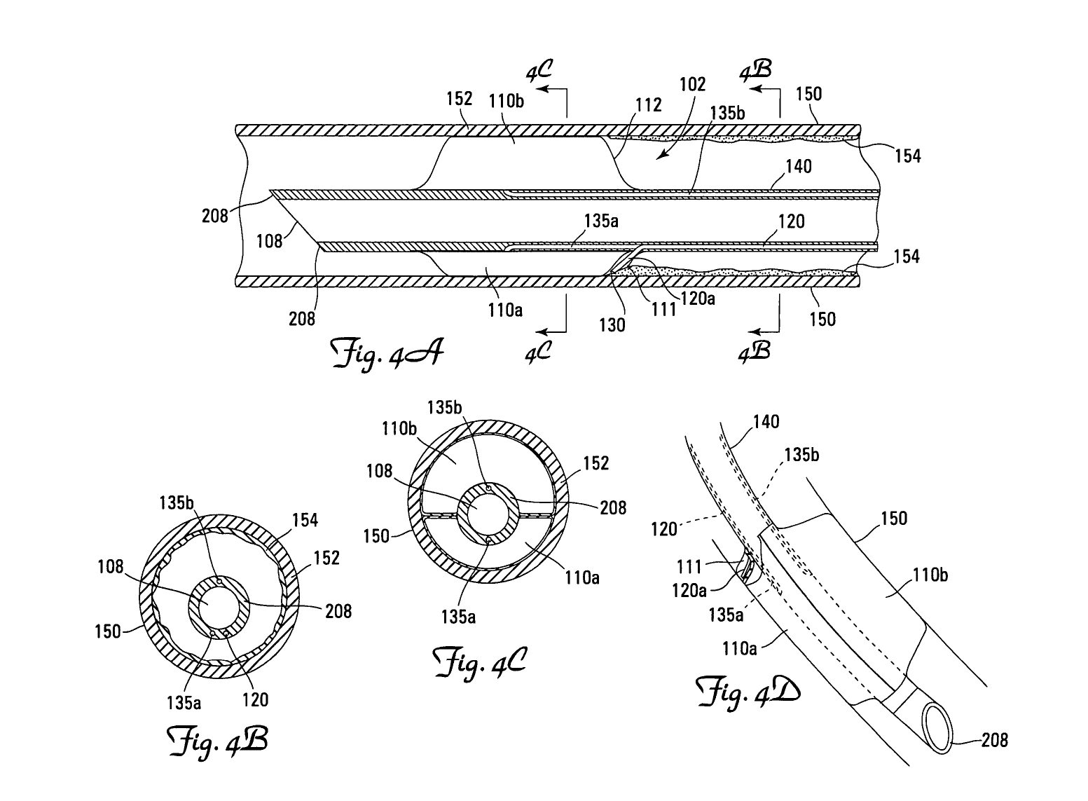

FIGS. 5A to 5C show an endotracheal tube 140 that is similar to that shown in FIG. 4A, except that inflatable cuff portions 110a, 110b are separated by pressure relief valves 160 and there is only one cuff lumen 135. Cuff lumen 135 extends to inflatable cuff portion 110a. Cuff portion 110a is inflated first and once the pressure in cuff portion 110a is high enough to open the pressure relief valves 160, cuff portion 110b is inflated. The reason for providing two inflatable portions is to insure that the cuff inflates without wrinkles or creases near the inflation port. Wrinkles or creases could allow fluid to pass by the cuff and not be removed through the pickup port 130. Because inflatable portion 110a inflates before inflatable portion 110b, any wrinkles or creases will be displaced to inflatable portion 110b. This method of inflation insures that fluid pickup port 130 is properly positioned relative to the inner wall 152 of the patient's trachea.

The distal portion of fluid removal lumen 120 exits the wall 208 of the endotracheal tube 140 above the inflatable cuff 110a (the distal portion of the fluid removal lumen that extends from the wall 208 of the trachea tube is designated 120a) and terminates in fluid pickup port 130, which is located immediately above the proximal portion of the inflatable cuff 110a. The fluid pickup port 130 is adjacent to the wall of the trachea 150 (when the inflatable cuff 110 is inflated as shown). The inner wall 152 of the patient's trachea 150 is coated with mucus 154. A cuff lumen 135 extends through endotracheal tube assembly 102 from cuff 110a to the proximal portion of endotracheal tube assembly 102.

In the example of FIG. 5A, fluid pickup port 130 is located near the distal end of endotracheal tube assembly 102 and above the V-shaped depression 111 of the inflatable cuff 110a. Therefore, in this example, when the distal end of endotracheal tube assembly 102 is introduced into a patient's trachea, fluid pickup port 130 is located within the patient at the V-shaped depression 111 of the inflatable cuff 110a. In this example, the fluid pickup port 130 wicks fluid into the port 130. In another embodiment of the invention, the fluid pickup port 130 does not exhibit significant wicking action and the fluid is removed by a vacuum pump. In this embodiment, low volume pump 132 is replaced with a vacuum pump.

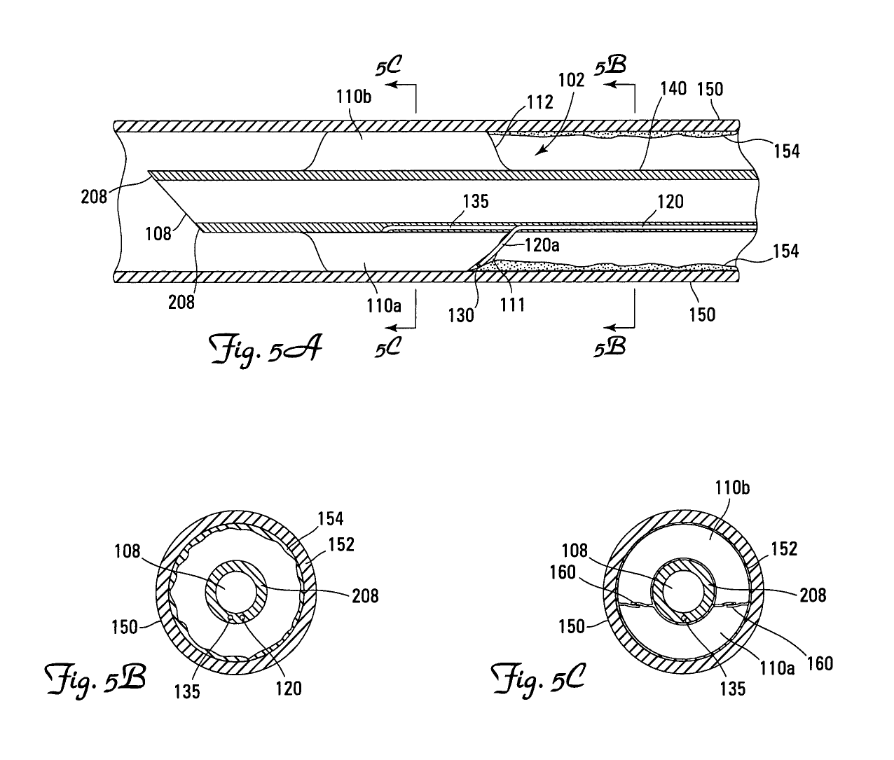

FIG. 6 is similar to FIG. 5A except there is an inflatable cuff 110, without separate portions, and the entire inflatable cuff is slanted relative to the longitudinal axis of the endotracheal tube 140. When inflated, the inflatable cuff 110 is slanted such that entire proximal surface 112 of the inflatable cuff is slanted relative to the longitudinal axis of the endotracheal tube 140. In one embodiment, the proximal surface 112 of the inflatable cuff and the longitudinal axis of the endotracheal tube meet at an angle of less than 90 degrees. This slanted configuration insures that fluid pickup port 130 is properly positioned relative to the inner wall 152 of the patient's trachea and helps to direct the fluid and/or mucus to the fluid pickup port 130.

The distal portion of fluid removal lumen 120 exits the wall 208 of the endotracheal tube 140 above the inflatable cuff 110 (the distal portion of the fluid removal lumen that extends from the wall 208 of the trachea tube is designated 120a) and terminates in fluid pickup port 130, which is located immediately above the proximal portion of the inflatable cuff 110. The fluid pickup port 130 is adjacent to the wall of the trachea 150 (when the inflatable cuff 110 is inflated as shown). The inner wall 152 of the patient's trachea 150 is coated with mucus 154. A cuff lumen 135 extends through endotracheal tube assembly 102 from cuff 110 to the proximal portion of endotracheal tube assembly 102. The cuff lumen and the fluid removal lumen can also be inside the air passage 108 or outside of the wall 208 of the tracheal tube.

In this example, the fluid pickup port 130 wicks fluid into the port 130. In another embodiment of the invention, the fluid pickup port 130 does not exhibit significant wicking action and the fluid is removed by a vacuum pump. In this embodiment, low volume pump 132 is replaced with a vacuum pump.

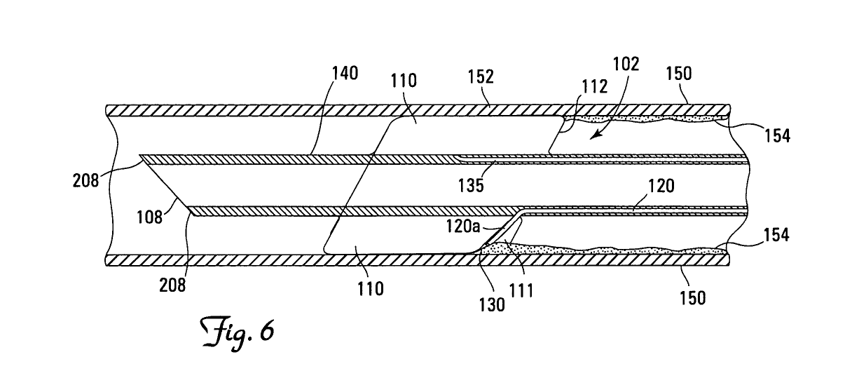

FIG. 7A shows an endotracheal tube 140 that is similar to that shown in FIG. 6, except that the embodiment of FIG. 7A includes reinforcing element 170. Reinforcing element 170 is less elastic than inflatable cuff 110 and is preferably made of a semi-rigid polymer such as DACRON-reinforced silicone rubber. Reinforcing element 170 helps to insure that the cuff inflates without wrinkles or creases near the pickup port 130 and that fluid pickup port 130 is properly positioned relative to the inner wall 152 of the patient's trachea. FIG. 7B is a cross section of FIG. 7A and shows the orientation of reinforcing element 170.

In an alternative embodiment, the same effect of the reinforcing element could be achieved by making the entire inflatable cuff 110 out of a semi-rigid polymer such as DACRON-reinforced silicone rubber. Such an embodiment can be illustrated as shown in FIG. 6.

In another embodiment, a portion of the inflatable cuff could be made of a semi-elastic material that expands only a predetermined distance from the wall 208 of the tracheal tube 102. The predetermined distance is chosen so that when the cuff is inflated, the fluid pickup port is a predetermined distance from the tracheal wall. Appropriate materials for the semi-elastic portion of the cuff include woven and nonwoven fabrics. Nonwoven nylon can be used. As the cuff is inflated, the semi-elastic portion reaches its limit of expansion early. Then the non-reinforced elastic portion of the cuff continues to expand to fill the trachea. The fluid pickup port is always located at the same position relative to the tracheal wall and the endotracheal tube.

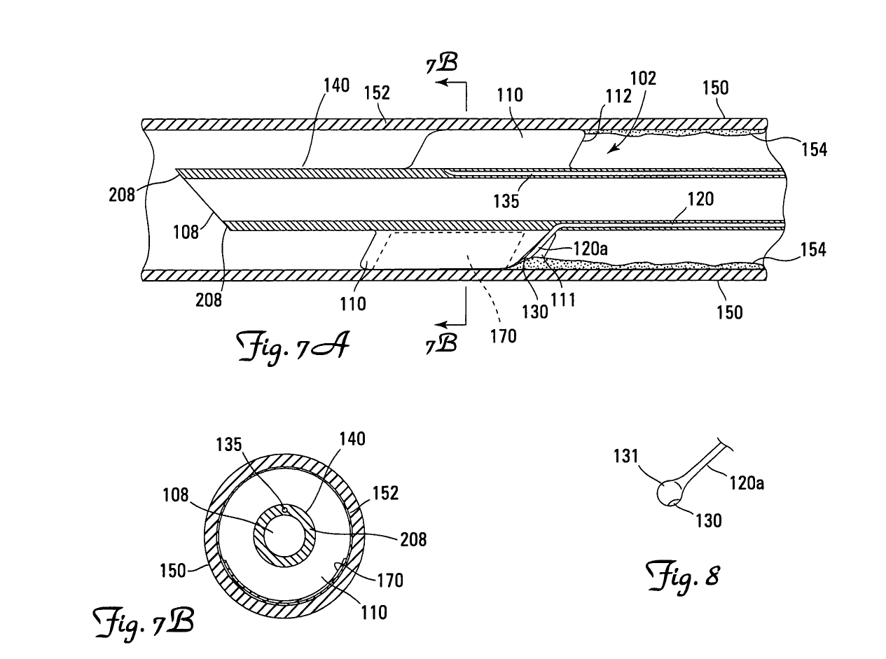

FIG. 8 shows a detail of one embodiment of a fluid pickup port 130, having fluid removal lumen 120a and rounded portion 131.

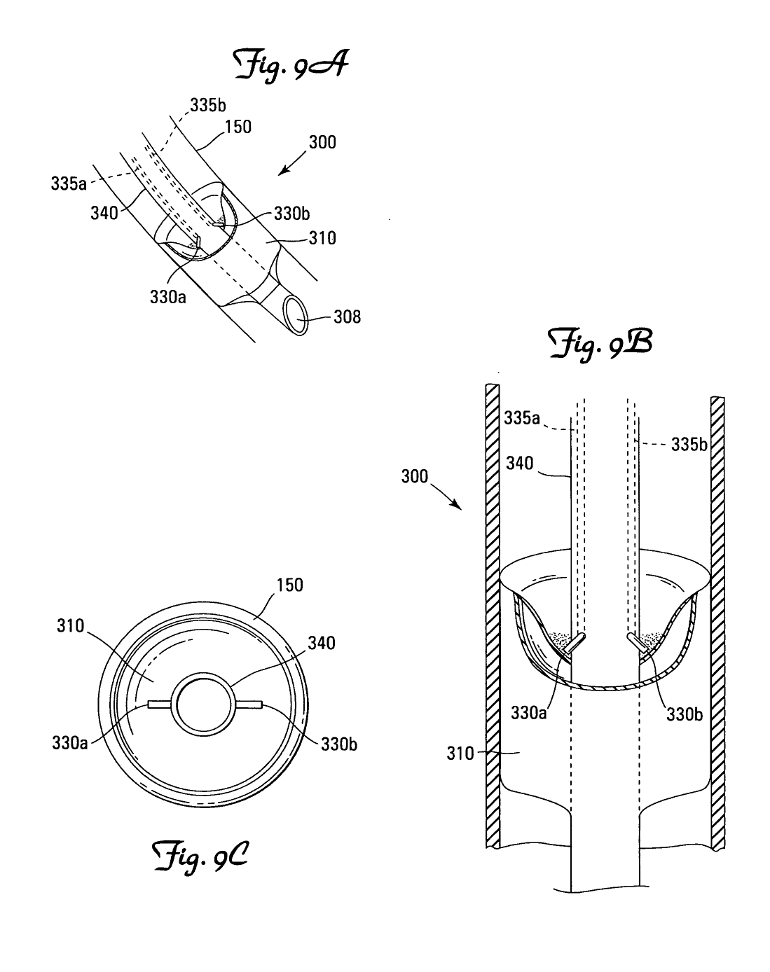

FIGS. 9A, 9B, and 9C show an embodiment of an endotracheal tube assembly 300 including an endotracheal tube 340 and an air passage 108, extending longitudinally between the distal and proximal ends of endotracheal tube assembly 300. A bladder-like inflatable cuff 310 (or other seal) is located about the outer circumference of endotracheal tube assembly 300 near its distal end. The inflatable cuff 310 is cup-shaped and oriented so that mucus from above the cuff pools on the proximal surface 311 of the inflatable cuff. The endotracheal tube assembly 300 is shown in place in trachea 150

Fluid pickup ports 330a, 330b are disposed above the inflatable cuff 310 and transport fluid through fluid removal lumens 335a and 335b. A different number of fluid removal lumens 335 may be provided, for example, corresponding to a different number of fluid pickup ports 330. This increases the number of surface energy assisted mucus collection sites. Such fluid pickup ports may be located in many different possible configurations. In this example, the fluid pickup port 330a, 330b wicks fluid into the ports 330a, 330b. In another embodiment of the invention, the fluid pickup ports 330a, 330b do not exhibit significant wicking action and the fluid is removed by a vacuum pump. In this embodiment, a low volume pump is replaced with a vacuum pump.

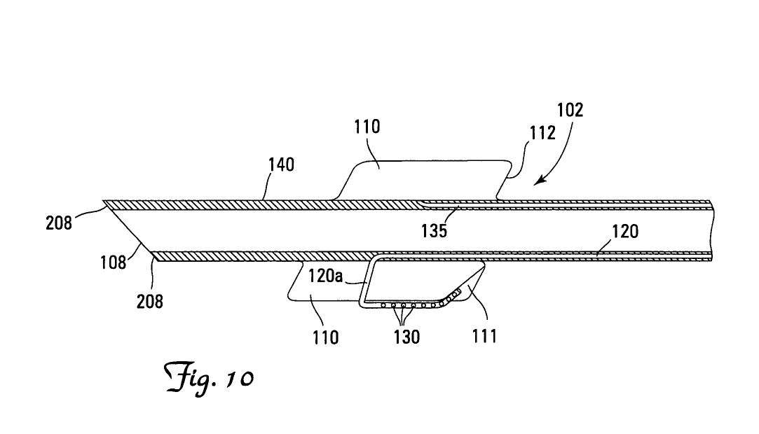

FIG. 10 is similar to FIG. 6 except that the fluid removal lumen 120 includes multiple fluid pickup ports 130 and the fluid removal lumen 120 exits the inflatable cuff distal of the proximal surface 112 and then proceeds proximally along the exterior of the cuff 110 to V-shaped depression 111. When the cuff 110 is expanded, many of the fluid pickup ports 130 are blocked by the cuff and the tracheal wall so only the most proximal pickup ports 130 can wick the fluid. The advantage of this design is that there are multiple fluid pickup ports available so if one or more fluid pickup ports 130 are blocked, the other ports 130 can still remove fluid. In another embodiment, the fluid removal lumen 120 could exit the wall 208 of the endotracheal tube distal of the inflatable cuff 110 and then proceed proximally along the exterior of the cuff 110 to the V-shaped depression 111.

The above descriptions are provided for the purpose of describing embodiments of the invention and are not intended to limit the scope of the invention in any way. It will be apparent to those skilled in the art that various modifications and variations can be made in the endotracheal tube having above the cuff drainage without departing from the spirit or scope of the invention. Thus, it is intended that the present invention cover the modifications and variations of this invention provided they come within the scope of the appended claims and their equivalents.

What is claimed is:

1. A system comprising: an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated.

2. The system of claim 1, wherein the at least one wicking fluid pickup port is disposed from 0.2 to 2 mm from the inner tracheal wall when the distal end of the elongated member is positioned within a trachea of a person and the inflatable cuff is inflated.

3. The system of claim 1, wherein the at least one wicking fluid pickup port is disposed immediately adjacent to the inner tracheal wall when the distal end of the elongated member is positioned within a trachea of a person and the inflatable cuff is inflated.

4. The system of claim 1, wherein the at least one wicking fluid pickup port is disposed immediately above the proximal surface of the cuff.

5. The system of claim 1, wherein the at least one wicking fluid pickup port is disposed on the proximal surface of the cuff.

6. The system of claim 1, wherein at least a portion of the proximal surface is slanted relative to a longitudinal axis of the elongated member to direct fluid toward the at least one wicking fluid pickup port.

7. The system of claim 6, wherein the entire proximal surface of the inflatable cuff is slanted relative to a longitudinal axis of the elongated member to direct fluid toward the at least one wicking fluid pickup port.

8. The system of claim 7, wherein the entire proximal surface of the cuff and the longitudinal axis of the elongated member meet at an angle of less than 90 degrees.

9. The system of claim 7, wherein the entire proximal surface of the cuff and the longitudinal axis of the elongated member meet at an angle of less than 80 degrees.

10. The system of claim 6, wherein the at least a portion of the proximal surface slanted relative to a longitudinal axis of the elongated member forms a V-shaped depression.

11. The system of claim 7, wherein the proximal surface comprises a V-shaped depression to direct fluid toward the at least one wicking fluid pickup port.

12. The system of claim 1, wherein the cuff comprises a reinforcing element.

13. The system of claim 1, wherein the cuff comprises a semi-rigid polymer.

14. The system of claim 1, wherein the cuff is made of a semi-rigid polymer.

15. The system of claim 1, wherein a portion of the inflatable cuff is made of a semi-elastic material that expands only a predetermined amount.

16. The system of claim 15, wherein the predetermined amount of expansion places the wicking fluid pickup port a predetermined distance from the tracheal wall when the cuff is inflated.

17. The system of claim 15, wherein the semi-elastic material is a woven or non-woven fabric.

18. The system of claim 1, wherein the elongated member comprises an airflow lumen extending longitudinally from at or near the distal end of the elongated member to or near the proximal end of the elongated member.

19. The system of claim 18, wherein the airflow lumen is sized to provide adequate ventilation to at least one lung of a person when the airflow lumen is coupled, at or near the proximal end of the elongated member, to a mechanical ventilator.

20. The system of claim 1, further comprising a cuff lumen, coupled in fluid communication with the inflatable bladder, the cuff lumen extending longitudinally to or near the proximal end of the elongated member.

21. The system of claim 20, wherein the elongated member comprises a hollow tube including a ventilation airflow lumen extending between the distal end of the elongated member and the proximal end of the elongated member, and in which at least a portion of the cuff lumen extends longitudinally through a sidewall portion of the hollow tube.

22. The system of claim 20, wherein the elongated member comprises a hollow tube including a ventilation airflow lumen extending between the distal end of the elongated member and the proximal end of the elongated member, and in which at least a portion of the cuff lumen extends longitudinally within the ventilation airflow center lumen.

23. The system of claim 20, wherein the elongated member comprises a hollow tube including a ventilation airflow lumen extending between the distal end of the elongated member and the proximal end of the elongated member, and in which at least a portion of the cuff lumen extends longitudinally outside the hollow tube.

24. The system of claim 1, wherein the inflatable cuff comprises a single inflatable bladder.

25. The system of claim 1, wherein the inflatable cuff comprises two inflatable bladders.

26. The system of claim 25, wherein the two inflatable bladders are coupled in fluid communication to first and second cuff lumens, the first and second cuff lumens extending longitudinally to or near the proximal end of the elongated member.

27. The system of claim 25, wherein a first inflatable bladder is coupled in fluid communication to a cuff lumens, the lumen extending longitudinally to or near the proximal end of the elongated member, and the second inflatable bladder is coupled in fluid communication with the first inflatable bladder by one or more pressure relief valves that open when the pressure in the first inflatable bladder exceeds a certain pressure.

28. The system of claim 1, wherein the at least one wicking fluid pickup port includes at least one of a size, shape, and material characteristic that obtains a surface energy capable of assisting in introducing mucus into the at least one wicking fluid pickup port.

29. The system of claim 1, further including a pump coupled in fluid communication with the at least one lumen that is in fluid communication with the at least one wicking fluid pickup port.

30. The system of claim 29, wherein at least one lumen that is in fluid communication with the at least one wicking fluid pickup port comprises a portion that extends within an interior portion of the inflatable cuff.

31. The system of claim 30, wherein a portion of the at least one lumen that is in fluid communication with the at least one wicking fluid pickup extends distally past the at least one wicking fluid pickup port.

32. The system of claim 29, wherein the pump comprises a peristalsis pump.

33. The system of claim 1, further including a holding receptacle coupled in fluid communication with the at least one lumen that is in fluid communication with the at least one wicking fluid port.

34. The system of claim 1, wherein the elongated member is sized and shaped to be inserted through an airflow passage of a tracheal tube assembly to a desired bronchial tube of the subject.

35. The system of claim 1, wherein the wicking fluid pickup port has a rounded tip.

36. The system of claim 1, wherein the elongated member comprises a reference mark to assist in proper placement of the elongated member.

37. The system of claim 1, wherein the system comprises two or more wicking fluid pickup ports.

38. The system of claim 37, wherein the lumen coupled to the at least one wicking fluid pickup port exits the inflatable cuff distal of the proximal surface of the inflatable cuff.

39. The system of claim 1, wherein the at least one wicking fluid pickup port is disposed immediately above or on the proximal surface of the cuff and an area of the cuff surrounding the at least one wicking fluid pickup port is hydrophilic.

40. The system of claim 39, wherein an area of the cuff distant from the at least one wicking fluid pickup port is hydrophobic.

41. A system comprising: an elongated member, sized and shaped to be inserted within a subject's trachea, the elongated member comprising proximal and distal ends; a seal, extending around the elongated member at or near the distal end of the elongated member; at least one fluid pickup port, positioned near the distal end of the elongated member, the at least one fluid pickup port located more proximal than at least a portion of the seal; and at least one lumen, coupled in fluid communication with the at least one fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated.

42. The system of claim 41, further comprising a vacuum pump coupled in fluid communication with the at least one lumen that is in fluid communication with the at least one wicking fluid pickup port.

43. A system comprising: an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, the inflatable cuff being cup-shaped and oriented so that the proximal surface directs fluid toward the at least one wicking fluid pickup port.

44. A method comprising:
inserting into a subject's trachea an elongated member, sized and shaped to be inserted within a subject's trachea, the elongated member comprising proximal and distal ends; a seal, extending around the elongated member at or near the distal end of the elongated member; at least one wicking fluid pickup port, positioned near the distal end of the elongated member, the at least one wicking fluid pickup port located more proximal than at least a portion of the seal; and at least one lumen, coupled in fluid communication with the at least one wicking fluid pickup port and extending toward the proximal end of the elongated member, the seal comprising an inflatable cuff having a proximal surface, wherein the at least one wicking fluid pickup port is disposed near an inner tracheal wall when the distal end of the elongated member is positioned within a trachea of a person and the inflatable cuff is inflated, and wherein the elongated member comprises an airflow lumen extending longitudinally from at or near the distal end of the elongated member to or near the proximal end of the elongated member;
inflating the inflatable cuff to obstruct airflow at a first location outside of the elongated member and inside the trachea;
ventilating at least one of the subject's lungs through the elongated member;
wicking fluid, at a location that is more proximal than the first location; and
drawing the wicked fluid out of the subject.

45. The method of claim 44, wherein the drawing the wicked fluid out of the subject includes using a peristalsis pump to provide a pressure for drawing the wicked fluid out of the subject.

46. The method of claim 44, wherein the drawing the wicked fluid out of the subject includes matching a flow rate at which the wicked fluid is drawn out of the subject to a mucus generation rate of the subject.
